(19) United States
(12) Patent Application Publication
(10) Pub. No.: US 2023/0381783 A1
(43) Pub. Date: Nov. 30, 2023

(54) DEVICES FOR OSCILLATING A FLUID SAMPLE

(71) Applicant: Hewlett-Packard Development Company, L.P., Spring, TX (US)

(72) Inventors: Carson Denison, Corvallis, OR (US); Erik D. Torniainen, Corvallis, OR (US); Richard W. Seaver, Corvallis, OR (US)

(73) Assignee: Hewlett-Packard Development Company, L.P., Spring, TX (US)

(21) Appl. No.: 18/032,543

(22) PCT Filed: Oct. 19, 2020
(86) PCT No.: PCT/US2020/056348

§ 371 Date: Apr. 18, 2023

(51) Int. Cl. B01L 7/00; C12Q 1/686

(52) U.S. Cl. CPC B01L 7/525; C12Q 1/686; B01L 2300/1827; B01L 2400/0442; B01L 3/502784

(57) ABSTRACT

In one example in accordance with the present disclosure, a device for conducting a reaction with a fluid sample is described. The device includes: a microfluidic channel; a number of heating elements along the microfluidic channel; and an inertial pump at each of opposite ends of the microfluidic channel to oscillate the fluid sample along the microfluidic channel.

### BACKGROUND

In a number of contexts, there is a need in the life sciences to quickly and accurately cycle small volumes of a fluid sample between temperatures so as to conduct a desired reaction. In healthcare, such a reaction may be used to diagnose a number of different conditions in a patient from which the fluid sample has been extracted. In particular, the ability to cycle fluid repeatedly between different temperatures is a bottleneck for the Polymerase Chain Reaction (PCR). This reaction duplicates DNA and is relevant in a number of medical testing scenarios.

### BRIEF DESCRIPTION OF THE DRAWINGS

The accompanying drawings illustrate various implementations of the principles described herein and are a part of the specification. The illustrated implementations are merely examples and do not limit the scope of the claims.

FIG. 1 is a diagram of an illustrative reaction device, consistent with the disclosed implementations.

FIG. 2A is a diagram of an illustrative reaction device, consistent with the disclosed implementations.

FIG. 2B is a diagram of an illustrative reaction device, consistent with the disclosed implementations.

FIG. 3 is a diagram of an illustrative reaction device, consistent with the disclosed implementations.

FIG. 4 is a diagram of an illustrative reaction device, consistent with the disclosed implementations.

FIG. 5 is a flowchart illustrating a method of oscillating fluid in a reaction device, consistent with the disclosed implementations.

FIG. 6 is a flowchart illustrating a method of oscillating fluid in a reaction device, consistent with the disclosed implementations.

FIG. 7 is a flowchart illustrating a method of oscillating fluid in a reaction device, consistent with the disclosed implementations.

FIG. 8A is a flowchart illustrating a method of oscillating fluid in a reaction device, consistent with the disclosed implementations.

FIG. 8B is a flowchart illustrating a method of oscillating fluid in a reaction device, consistent with the disclosed implementations.

FIG. 9 is a diagram of an illustrative reaction device, consistent with the disclosed implementations.

FIG. 10 is a diagram of an illustrative reaction device, consistent with the disclosed implementations.

FIGS. 11A-11F is a diagram of an illustrative method of forming a reaction device, consistent with the disclosed implementations.

FIG. 12 is a profile diagram of an illustrative reaction device, consistent with the disclosed implementations.

Throughout the drawings, identical reference numbers designate similar, but not necessarily identical, elements.

### DETAILED DESCRIPTION

As noted above, in the life sciences, including in healthcare, there may be a desire to quickly and accurately cycle small volumes of a fluid sample between temperatures so as to conduct a desired reaction. In particular, the ability to cycle fluid repeatedly between different temperatures is used in the Polymerase Chain Reaction (FOR) which duplicates deoxyribonucleic acid (DNA) and is relevant in a number of medical testing scenarios.

Specifically, a PCR operation makes millions to billions of copies of a specific DNA sample. As such, a scientist may take a small sample of DNA and amplify it to a large number of copies, such that it may be studied in detail. During PCR, a sample is quickly heated to around 100 degrees Celsius (C) and then cooled to around 50 degrees C. This process is repeated tens of times.

While operations such as PCR greatly enhance the ability of scientists to carry out a variety of experiments, some advancements to devices that carry out PCR may further increase its efficacy and use in scientific laboratories and doctors' offices.

For example, some systems may not be able to change the fluid temperature rapidly. For example, some PCR devices take around 30 minutes to 1 hour to complete the PCR cycles. Moreover, for PCR reactions, fluid flows through regions of specified temperatures, for example between 20 and 40 times, to complete the reaction. Accordingly, some PCR devices are large as they are designed to have long enough channels to contain 20 to 40 repetitions of each temperature zone.

Accordingly, the following description describes a device for addressing these and other issues. Specifically, the present specification describes devices and methods that rapidly and accurately change the temperature of small volumes of fluid and may be particularly implemented in a PCR operation where fluid temperature is to be changed multiple times. Specifically, the devices of the present specification include a microfluidic channel with reservoirs on either end. Disposed along the microfluidic channel are heating elements such as resistors that can be tuned to heat the fluid to different temperatures. Inertial pumps at either end of the microfluidic channel move the fluid back and forth past the heating elements, which heating elements then cyclically heat the fluid, as in a PCR operation.

In an example the device includes a microfluidic channel; a number of heating elements along the microfluidic channel; and an inertial pump at each of opposite ends of the microfluidic channel to oscillate the fluid sample along the microfluidic channel.

In an example, the device includes a fluid reservoir at each end of the microfluidic channel. In this example, one of the inertial pumps is located at each interface between the microfluidic channel and one of the fluid reservoirs.

In an example, the device includes a fluid reservoir. In this example, the microfluidic channel has opposite ends that are both in fluid connection with the fluid reservoir. Still in this example, one of the inertial pumps is located at each interface between the microfluidic channel and the reservoir. In an example, the heating elements may be thin-film resistors. Thin film resistors are a type of resistor that includes a thin resistive layer disposed on a substrate. For example, the thin-film resistor may have a thickness of between 0.05 μm and 0.5 μm. In some examples, the thickness can be around 0.1 μm. A thin film resistor may be formed by depositing a metal layer on a substrate. This metal layer may be formed of materials such as chromium, nickel, and nichrome. The metallic layer may be patterned using photolithography and subtractive processes to tune a resistive value.

As described above, the reaction conducted in the device may be a Polymerase Chain Reaction (PCR). In this example, the number of heating elements is three where each heating element is to maintain a different temperature in the PCR. In such an example, a first heating element corresponds to a denature phase of the PCR, a second heating element corresponds to an anneal phase of the PCR, and a third heating element corresponds to an extension phase of the PCR.

In an example, only one heating element is disposed along the microfluidic channel. In an example, the device includes an injector to insert a bubble of immiscible fluid on either side of the fluid sample in the microfluidic channel. Still further, in an example each inertial pump may be a thermal inkjet pump.

The present description also describes a method of performing a Polymerase Chain Reaction (PCR). According to the method, a fluid sample is introduced into a microfluidic channel. The fluid sample is oscillated in two directions in the microfluidic channel. A number of heating elements disposed along the microfluidic channel repeatedly heat the fluid sample.

In an example, the fluid sample is separated with a bubble of immiscible fluid on either side of the fluid sample within the microfluidic channel.

In an example, a first heating element is heated to a denature temperature and a second heating element is heated to an anneal temperature. In this example, the anneal temperature is lower than the denature temperature. Still in this example, a third heating element is heated to an extension temperature, which extension temperature is between the denature and anneal temperatures.

In a particular example, a first of the heating elements is heated to a denature temperature for a denature phase of the PCR. In this particular example none of the heating elements are heated during an anneal phase of the PCR and all of the heating elements are heated to an extension temperature during an extension phase of the PCR. In this particular example, after heating the fluid sample with the first heating element at the denature temperature, all heating elements are turned off and the fluid sample is moved in the microfluidic channel. After the anneal phase of the PCR, the fluid in the microfluidic channel is again moved and all the heating elements are heated to the extension temperature. After the extension phase of the PCR, the fluid sample is returned to an initial position.

The present description also describes a Polymerase Chain Reaction (PCR) device for conducting a PCR on a fluid sample. In various examples, the PCR device includes a microfluidic channel; a number of heating elements along the microfluidic channel; an inertial pump at each of opposite ends of the microfluidic channel to oscillate the fluid sample along the microfluidic channel; a side channel fluidly connected to the microfluidic channel for introducing the fluid sample into the microfluidic channel; and a pump at the junction of the microfluidic channel and side channel to provide a microfluidic valve between the side channel and microfluidic channel.

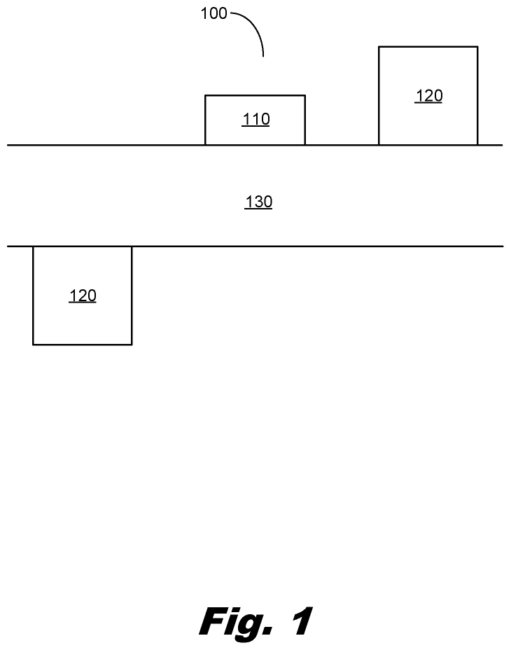

FIG. 1 is a diagram of an illustrative reaction device 100, consistent with the disclosed implementations. As shown in FIG. 1, the reaction device 100 includes a microfluidic channel 130; a number of heating elements 110 along the microfluidic channel 130; and an inertial pump 120 at each of opposite ends of the microfluidic channel 130 to oscillate a fluid sample along the microfluidic channel 130.

In some examples, the reaction device 100 is a microfluidic structure. Such microfluidic structures may be a few square millimeters to a few square centimeters, and may provide efficient small-scale functionality. In other words, the components, i.e., microfluidic channel 130, heating elements 110, and inertial pumps 120 may be microfluidic structures. A microfluidic structure is a structure of sufficiently small size (e.g., of nanometer sized scale, micrometer sized scale, millimeter sized scale, etc.) to facilitate conveyance of small volumes of fluid (e.g., picoliter scale, nanoliter scale, microliter scale, milliliter scale, etc.). As a particular example, the microfluidic channel 130 may be, for example, about 1-2 millimeter (mm) in length. The width of the channel 130 may be, for example, 20 microns. A 20 micron wide infinite planar channel has the following pressure drops for a fluid of 1 centipoise (cP): 0.75 millibar per millimeter (mbar/mm) at 1 millimeter per second (mm/s); 7.5 mbar/mm at 10 mm/s, and 75 mbar/mm at 100 mm/s.

The reaction device 100 also includes a number of heating elements 110 along the microfluidic channel 130. The heating elements 110 may be used to raise the temperature of the fluid sample passing through the microfluidic channel 130. For example, as described above, in certain operations, such as PCR, it may be desirable to cyclically heat the fluid sample. In such an example, the heating elements 110 carry out such heating. In one example, each heating element 110 includes a thin-film resistor. That is the heating elements 110 may include a resistor formed on a substrate such as a layer of silicon material. While FIG. 1 depicts just one heating element 110 disposed along the microfluidic channel 130, in some examples there may be multiple heating elements 110 disposed along the microfluidic channel 130.

The reaction device 120 also includes an inertial pump 120 at each of opposite ends of the microfluidic channel 130 to oscillate the fluid sample along the microfluidic channel 130. The inertial pumps 120, when not operating, allow fluid to flow in the opposite direction from that which the inertial pump 120 acts. Accordingly, with two inertial pumps 120, that are alternately activated, fluid can by oscillated back and forth in the microfluidic channel 130. Specifically, the inertial pump 120 shown on the right can be operated to drive a fluid sample in the microfluidic channel 130 to the left in the drawing, while the inertial pump 120 on the left is inactive. Then, the inertial pump 120 shown on the left can be operated to drive the fluid sample through the microfluidic channel 130 to the right in the drawing. The fluid can also be left stationary when both inertial pumps 120 are inactive.

The inertial pumps 120 may be based on inkjet technology, that is the inertial pumps 120 may be thermal inkjet pumps. In such an implementation, the inertial pumps 120 include a drive element that forces fluid movement. The drive element could be a heater, such as a heating resistor, or a piezoelectric element. In an inertial pump 120 with a heating drive element, the heater, perhaps a resistor, heats the adjacent fluid. This causes the heated fluid to vaporize, creating a bubble that pushes on the fluid sample to pump the fluid in the desired direction. A single thermal inkjet pump with pressure head of 54 mbar can pump fluid at 210 mm/s in a 20×20 um microfluidic channel 130.

In a piezoelectric inertial pump, an electric field is alternately applied to the piezoelectric drive element. This causes the element to expand and contract, pushing on the fluid of the fluid sample to pump the fluid in a desired direction.

With regard to power needs, a thermal inkjet pump can move about 8 picoliter (pL) per pulse with about 1 microjoule of energy. At 8 pL/pulse, it would take 30 total pulses to do one cycle. This would use 1.2 milliJoules (mJ) total over 40 cycles. Assuming the total cycle time is 10 seconds (s), then that is 1.2 mJ/10 s=0.12 mW. Accordingly, each of 6 heating elements may run at ˜1 mW.

The number of heating elements 110 along the microfluidic channel 130 are not to be confused with a heater incorporated into one of the inertial pumps 120. For example, inertial pumps 120 may fire in 1 microsecond. Consequently, the corresponding thermal effect on the fluid is quite small. By comparison, the heating elements 110 are used to control the temperature of the fluid and impact the fluid thermally by actuating for longer than 1 microsecond. With a thermal inkjet pump and other inertial pumps 120, fluid can be pumped through the microfluidic channel 130 at tens of mm/s with a travel time between spaced heating zones of less than 0.1 seconds. The space between heating zones should have just slightly more than the same diffusion time as the space across the channel.

As will be described below, the reaction device 100 may include multiple heating elements 110. The number of heating elements 110 correspond to heating zones to change the temperature of the fluid sample in the microfluidic channel 130. In many reactions of interest that a user may want to conduct on the fluid sample, the fluid sample may need to experience different temperatures. Accordingly, the number of heating elements 110 could, in some examples, be a single heating element 110 that operates at different temperatures at different times as needed to produce the desired reaction in the fluid sample. In other examples, the number of heating elements 110 may include multiple heating elements 110 disposed along the microfluidic channel 130, with the different heating elements 110 operating at different temperatures. In this example, the fluid sample is moved through the microfluidic channel 130 by the inertial pumps 120 to the heating element 110 with the temperature needed next for the reaction or test being conducted on the fluid sample.

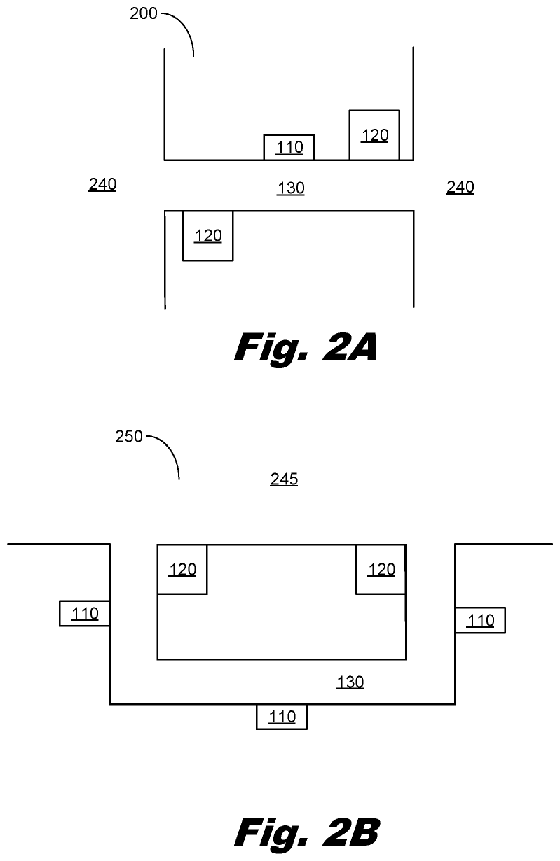

FIG. 2A is a diagram of an illustrative reaction device 200, consistent with the disclosed implementations. In the example shown in FIG. 2A, the reaction device 200 is similar to that shown in and described with regard to FIG. 1. However, in the example shown in FIG. 2A, the reaction device 200 includes a fluid reservoir 240 at each end of the microfluidic channel 130. In this example, as depicted in FIG. 2A, one of the inertial pumps 120 is located at each interface between the microfluidic channel 130 and one of the two fluid reservoirs 240. Consequently, the fluid sample may be contained in one or both of the fluid reservoirs 240 as needed. A quantity of the fluid sample for testing is then pumped to or from a fluid reservoir 240 by the inertial pumps 120 through the microfluidic channel 130. As above, the fluid sample in the microfluidic channel 130 is heated by the number of heating elements 110 to conduct a desired reaction.

In one example, the number of heating elements 110 may be three. In that example, each of the heating elements 110 may have a length of 50-100 microns along the microfluidic channel 130. This same distance may be provided between adjacent heating elements 110 and between heating elements 110 adjacent to one of the fluid reservoirs 240 and that fluid reservoir 240.

FIG. 2B is a diagram of an illustrative reaction device 250, consistent with the disclosed implementations. In the example shown in FIG. 2B, the reaction device 250 is similar to that shown in and described with regard to FIG. 1. However, in the example shown in FIG. 2B, the microfluidic channel 130 has a U-shape. That is, opposite ends of the microfluidic channel 130 are both in fluid connection with the fluid reservoir 245. In this example, one of the inertial pumps 120 is located at each interface between the microfluidic channel 130 and the fluid reservoir 245. A quantity of the fluid sample for testing is then pumped to or from the fluid reservoir 245 by the inertial pumps 120 through the microfluidic channel 130. As above, the fluid sample in the microfluidic channel 130 is heated by the number of heating elements 110 to conduct a desired reaction.

In this example, the heating elements 110 may each be a resistor or pair of resistors on opposite sides of the microfluidic channel 130. The heating element 130 on the left arm of the microfluidic channel may be held or operated at 50-60° C., which may represent an anneal temperature for a PCR operation. The heating element 10 at the center of the microfluidic channel 130 may be held or operated at 70-75° C., which may represent an extension temperature for the PCR operation. The heating element 110 on the right arm of the microfluidic channel 130 may be held or operated at 93-98° C., which may represent a denature temperature for the PCR operation. Note that these heating ranges are for a PCR example. However, any temperature ranges could be used depending on the reaction being conducted and the temperatures needed for that reaction. Each of the heating elements 110 may have a length of 50-100 microns along the microfluidic channel 130. The same distance may be provided between each heating element 110 edge adjacent to a corner of the U-shaped microfluidic channel 130 and that corner. This distance between each heating element 110 adjacent to the fluid reservoir 245 and the fluid reservoir 245 may be less than 200 microns.

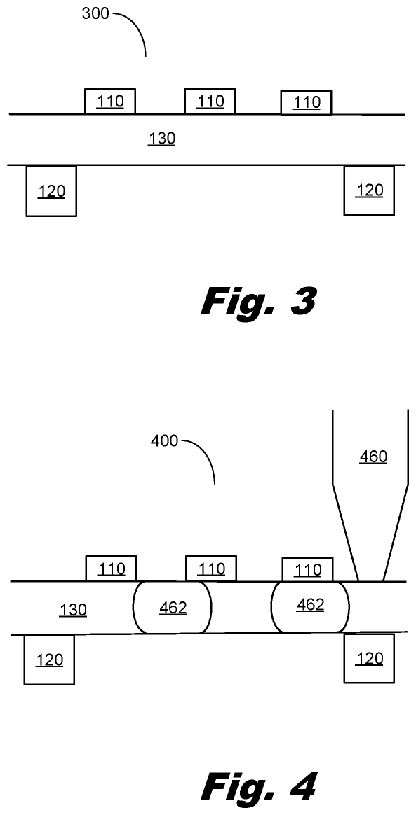

FIG. 3 is a diagram of an illustrative reaction device 300, consistent with the disclosed implementations. In the example shown in FIG. 3, the inertial pumps 120 again oscillate a fluid sample back and forth in the microfluidic channel 130.

In FIG. 3, three heating elements 110 are illustrated along the microfluidic channel 130. This configuration can be used in conducting a Polymerase Chain Reaction (PCR) in which the fluid sample is to be heated to three different temperatures during the reaction. In a PCR, these different temperatures or stages of the reaction are referred to as denature, anneal, and extension. In this example, a first heating element 110 may correspond to the denature phase of the PCR. That is, the first heating element 110 may heat the fluid to the denature temperature for the PCR, which denature temperature may be around 93-98° C. The second heating element 110 may correspond to an anneal phase of the PCR. That is, the second heating element 110 may heat the fluid to an anneal temperature for the PCR operation, which anneal temperature may be around 50-60° C. The third heating element 110 may correspond to an extension phase of the PCR. That is, the third heating element 110 may heat the fluid to an extension temperature for the PCR operation, which extension temperature may be around 70-75° C.

FIG. 4 is a diagram of an illustrative reaction device 400, consistent with the disclosed implementations. The example of FIG. 4 is similar to that of FIG. 3, discussed above, but also includes an injector 460.

This injector 460 is used to insert bubbles 462 of an immiscible fluid into the microfluidic channel 130 on either side of the fluid sample. The immiscible fluid can be a liquid or gas that is not reactive with the fluid sample. For example, the immiscible fluid could be air, some other gas, or oil. The injector 460 may include a nozzle to inject the immiscible fluid into the microfluidic channel 130. The injector 460 may be coupled to a reservoir that contains the immiscible fluid. Through operation of a pump, the immiscible fluid may be drawn from the reservoir, through a conduit to be injected into the microfluidic channel 130 from the nozzle.

In this way, as shown in FIG. 4, a quantity of the fluid sample can be isolated in the microfluidic channel 130 between two bubbles 462 of the immiscible fluid. The quantity of the fluid sample isolated between two bubbles 462 of immiscible fluid may be referred to as a fluid slug. Thus, the fluid slug may be run through a desired reaction while isolated from the larger quantity of the fluid sample in the device 400. Using the bubbles 462 of immiscible fluid on either side of the fluid slug also keeps the sample together in a limited volume instead of allowing the fluid sample to spread out during movement. In such an example, the system moves the fluid slug to an appropriate heating zone for each of the reaction steps, e.g., the three steps for a PCR.

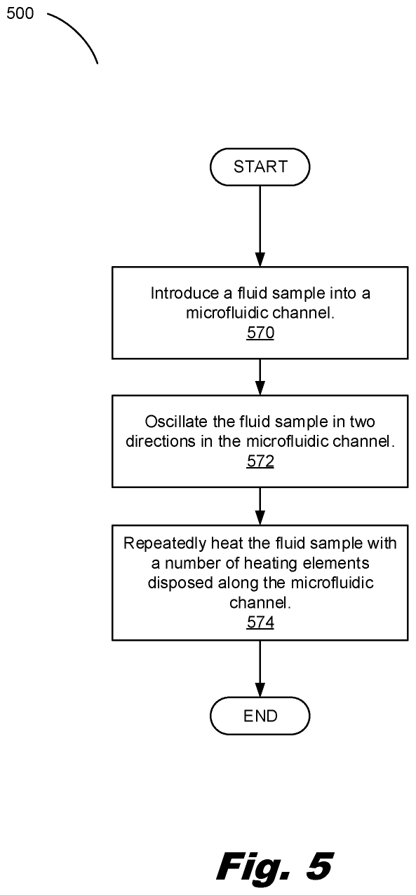

FIG. 5 is a flowchart illustrating a method 500 of operating a reaction device, consistent with the disclosed implementations. In FIG. 5, the illustrated method 500 includes: introducing 570 a fluid sample into a microfluidic channel; oscillating 572 the fluid sample in two directions in the microfluidic channel; and repeatedly heating 574 the fluid sample with a number of heating elements 110 disposed along the microfluidic channel. As noted above, one reaction that may be conducted according to this method 500 is the Polymerase Chain Reaction (PCR).

Specifically, the method 500 includes introducing 570 a fluid sample into a microfluidic channel 130. The fluid sample may be introduced via operation of the inertial pumps 120 to draw fluid from a fluid reservoir in fluid connection with the microfluidic channel 130. The fluid sample is then oscillated 572 in two directions within the microfluidic channel 130. For example, the inertial pumps 120 may be alternately activated. This alternate activation changes the direction of fluid flow through the microfluidic channel 130. For example, as described above in connection with FIG. 1, activation of one inertial pump 120 may move the fluid sample to the left. The second inertial pump 120, on an opposite end of the microfluidic channel 130, may then be activated to change the direction of fluid flow towards the right.

As the fluid passes through the microfluidic channel 130, the fluid sample is repeatedly heated 574 by the number of heating elements 110. This cyclic heating may, for example, effectuate the thermal cycling carried out during certain chemical operations, such as PCR.

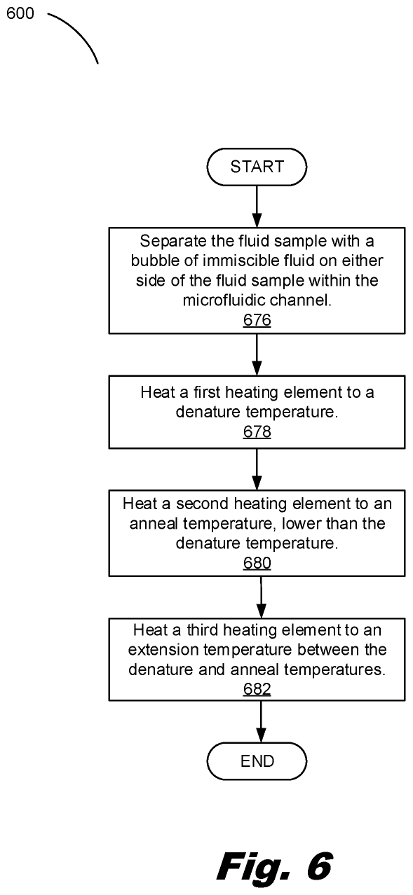

FIG. 6 is a flowchart illustrating a method 600 of operating a Polymerase Chain Reaction (PCR) device, consistent with the disclosed implementations. As shown in FIG. 6, the method 600 includes separating the fluid sample with a bubble 462 of immiscible fluid on either side of the fluid sample in the microfluidic channel 130 as discussed and illustrated above in connection with FIG. 4.

Next, the method 600 of FIG. 6 includes heating 678 a first of three heating elements 110 along the microfluidic channel 130 to a denature temperature of the PCR. During the denature phase of PCR, DNA may be separated into a single strand. A second of three heating elements 110 along the microfluidic channel 130 is heated to an anneal temperature of the PCR. The anneal temperature is lower than the denature temperature. During the anneal phase, DNA primers attach to the separated strands of DNA.

The method 600 also includes heating 682 a third of the three heating elements 110 to an extension temperature that is between the denature and anneal temperatures of the PCR. During the extension phase, replicated DNA strands are extended by the polymerase in a PCR master mix. The fluid sample is then oscillated back and forth through the microfluidic channel 130 to repeatedly expose the fluid sample to the denature, anneal and extension temperatures as needed to cause the PCR.

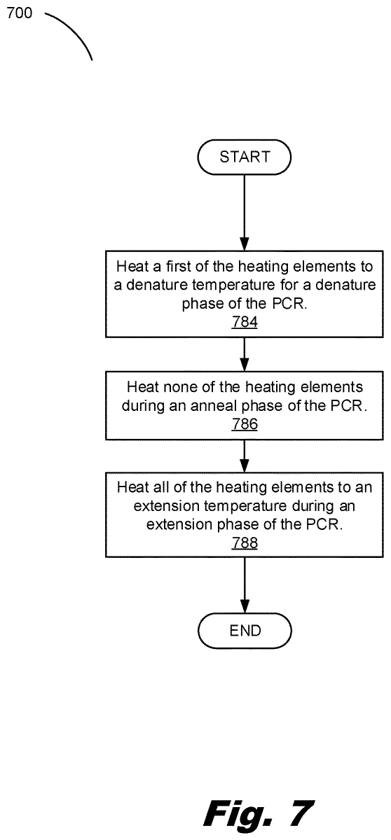

FIG. 7 is a flowchart illustrating a method 700 of operating a reaction device, consistent with the disclosed implementations. A configuration of multiple heating elements 110 along the microfluidic channel 130 can be operated in different ways to conduct a PCR in a fluid sample. FIG. 6 depicted one such configuration and FIG. 7 depicts another such configuration.

In the example of FIG. 7, a first of multiple heating elements 110 is heated 784 to a denature temperature for a denature phase of the PCR again to separate DNA into a single strand. After denature, none of the multiple heating elements 100 is heated or operated 786 to allow for an anneal phase of the PCR wherein DNA primers attach to the separated strands of DNA.

Lastly, all of the multiple heating elements 110 are heated 788 to the extension temperature such that replicated DNA strands are extended by the polymerase in a PCR master mix. This causes the extension phase of the PCR to occur in the fluid sample which may be heated by the multiple heating elements 110 for this phase.

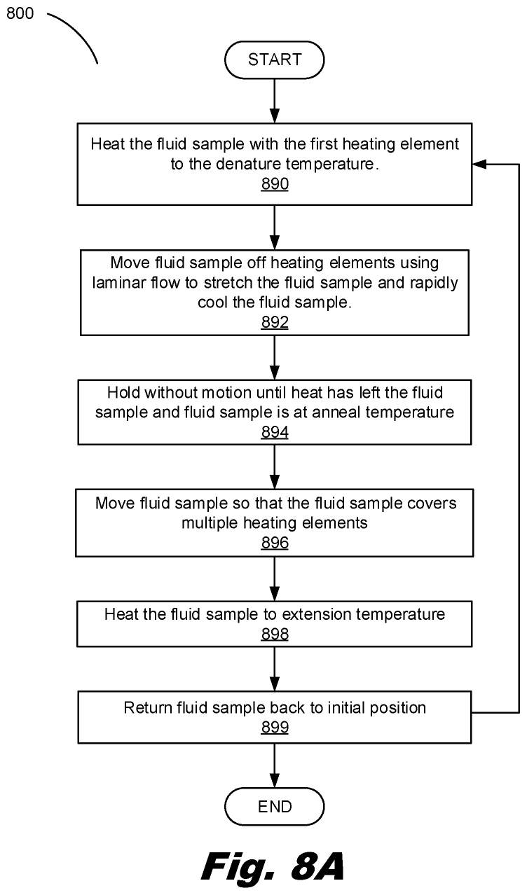

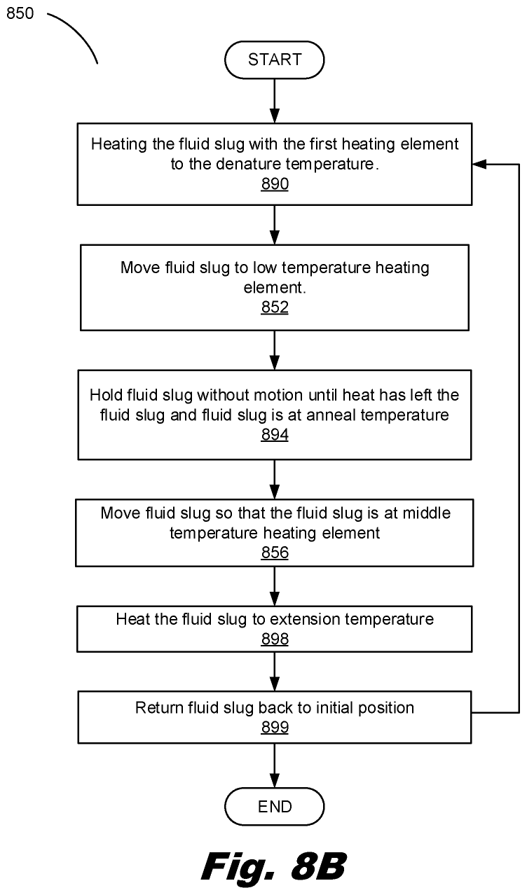

FIG. 8A is a flowchart illustrating a method 800 of operating a reaction device, consistent with the disclosed implementations. As noted above, a configuration of multiple heating elements 110 along the microfluidic channel 130 can be operated in different ways to conduct a PCR in a fluid sample. FIG. 8A depicts such an example.

In the example of FIG. 8A, the fluid sample is heated 890 with the first heating element 110 to the denature temperature, e.g., 97° C. The fluid sample is then moved 892 off the heating element 110 using laminar flow to stretch the fluid sample and rapidly cool the fluid sample. The stretched configuration of the fluid sample is produced by the laminar flow generated of the inertial pump 120 in the microfluidic channel 130. The stretched fluid sample has a higher surface area and thinner volume, which allows rapid cooling of the fluid sample. After heating the fluid sample with the first heating element 110 to the denature temperature, all heating elements are turned off and the fluid sample is held 894 without motion until heat has left the fluid sample and the fluid sample is at the anneal temperature, e.g., 50-60° C.

After the anneal phase of the PCR, the fluid sample is moved 896 so that the fluid sample covers multiple heating elements 110. All heating elements 110 then heat 898 the fluid sample to the extension temperature, e.g., 75° C. The fluid sample is then extended at the extension temperature. After the extension phase of PCR, the fluid sample is returned 899 back to the initial position. The cycle may then be repeated until the process is complete. In some examples, the cycle is repeated 15 to 30 times.

In this example, the fluid sample is stretched during cooling to the anneal temperature allowing rapid cooling due to the large surface area of the stretched sample. The heating to extension temperature may be performed over multiple heating elements 110 in a partially-stretched configuration. Finally, the heating to the denature temperature may be conducted in an unstretched configuration. This approach contrasts with the approach in FIG. 8B, below, which uses immiscible bubbles to contain the fluid sample in a fluid slug.

In the example of FIG. 8B, the method 850 uses the immiscible bubbles described above to contain a fluid slug in a smaller volume when pumped along the microfluidic channel 130. In this example, the fluid slug is heated 890 with a first of multiple heating elements 110 at the denature temperature, e.g. 97° C. After this heating, the fluid slug is moved 852 to a low temperature heating element 110, e.g., 50-60° C. The fluid slug is then held 894 without motion to allow annealing at the reduced temperature.

After the annealing phase, the fluid slug is moved 856 to a mid-temperature heating element 110 activated to the extension temperature, e.g. 75° C. The fluid slug is held at the mid-temperature heating element 110 to allow extension to occur.

After the extension phase, the inertial pumps 120 return 896 the fluid slug to the initial position, e.g., adjacent to the first of the heating elements 110. The cycle can then be repeated a number of times, as needed, to cause the PCR to occur in the fluid sample. In this example, the immiscible bubbles 462 keep the fluid slug contained.

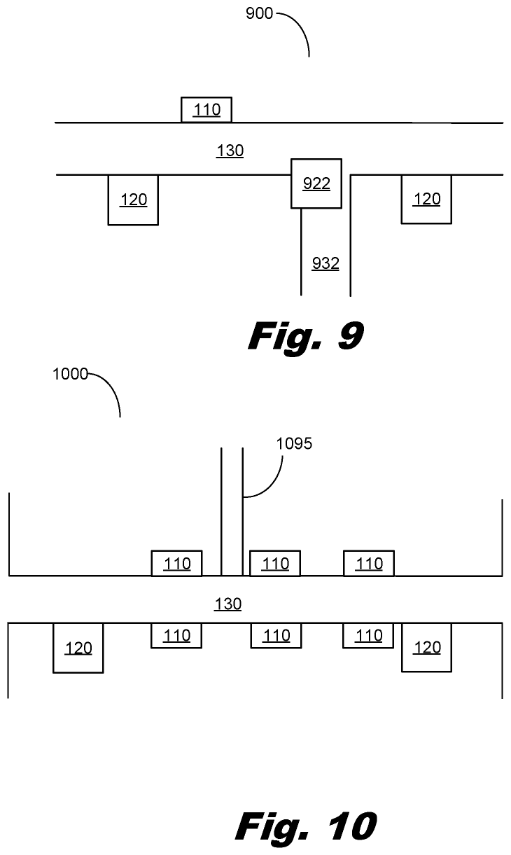

FIG. 9 is a diagram of an illustrative reaction device 900, consistent with the disclosed implementations. In the example of FIG. 9, two inertial pumps 120 are again provided to oscillate a fluid sample back and forth in the microfluidic channel 130. A number of heating elements 110 are again provided along the microfluidic channel 130 for heating the fluid sample as described herein.

In FIG. 9, a side channel 932 connects fluidly to the microfluidic channel 130. This side channel 932 may be used to introduce the fluid sample into the microfluidic channel 130. A pump 922 is provided at the confluence of the side channel 932 and the microfluidic channel 130. This pump 922 is used and operated as a valve. As with the inertial pumps 120, the pump 922 may be a thermal inkjet or piezoelectric pump to draw the fluid sample into the microfluidic channel 130.

In this example, the microfluidic channel 130 may be filed with water. The pump 922 may be used to draw a specific amount of fluid sample, e.g., a DNA rich fluid, from the side channel 932 into the microfluidic channel 130. In this way, the pump 922 can meter the amount of fluid sample introduced into the microfluidic channel 130. The pump 922 may also remove the fluid sample from the microfluidic channel 130 when the reaction is completed. In such an example, the pump 922 may include selectively-actuatable opposite elements to pump fluid in opposing directions.

FIG. 10 is a diagram of an illustrative reaction device 1000, consistent with the disclosed implementations. As shown in FIG. 10, the heating elements 110 may be disposed on opposite sides of the microfluidic channel 130. This may allow for faster heating and temperature control of the fluid sample in the microfluidic channel 130.

As depicted in FIG. 10, the reaction device 1000 may include a window 1095 that allows a user to observe the fluid sample as it flows through the microfluidic channel 130. This ability to visually observe the fluid sample may allow the user to determine the degree of reaction that has taken place and/or the result of the reaction in a way that allows the user to make a diagnosis or conclusion based on the reaction occurring in the fluid sample.

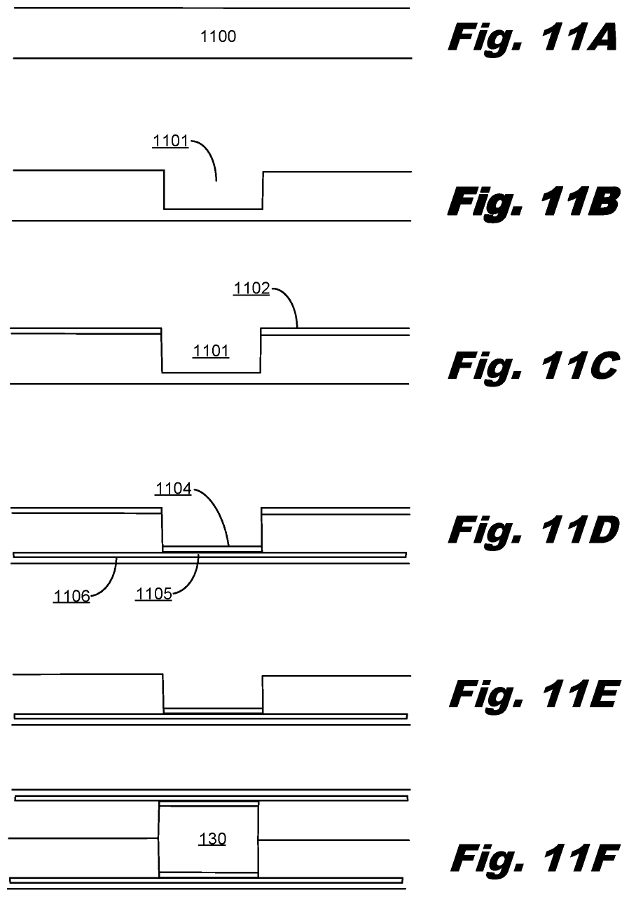

FIGS. 11A-11F are diagrams of an illustrative method of forming a reaction device, consistent with the disclosed implementations. FIG. 11A shows the use of a substrate 1100, for example, a silicon substrate, to form any of the illustrative devices described herein.

In FIG. 11B, a channel 1101 is etched or cut into the substrate 1100. In FIG. 11C, a mask 1102, e.g., photoresist, is applied on an upper surface of the substrate 1100. The mask 1102 protects the surface of the substrate 1100, but allows material deposition in the etched channel 1101.

In FIG. 11D, material for forming a resistor 1104, e.g., a metal, is deposited on the upper surface of the substrate 1100 and into the channel 1101 through the mask 1102. The opposite, lower surface of the substrate 1100 is removed to expose the underside of the resistor 1104. Conductive traces 1105 are then formed so that the resistor 1104 can be selectively energized to generate heat as desired. An insulating material 1106 is applied to the lower surface to electrically isolate the traces 1105 and resistor 1104.

In FIG. 11E, the mask 1102 and any material on the mask 1102 are removed. Then, in FIG. 11F, another unit made by the same steps illustrated above is attached to the first unit so as to complete the microfluidic channel 130.

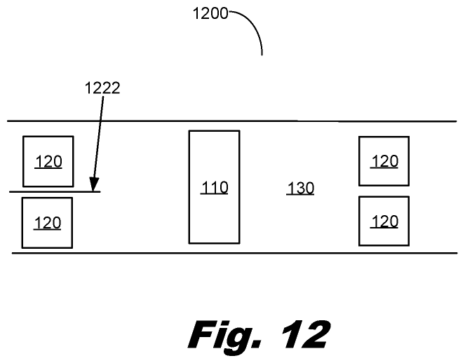

FIG. 12 is a profile diagram of an illustrative reaction device 1200, consistent with the disclosed implementations. As shown in FIG. 12, multiple inertial pumps 120 may be disposed on each end of the microfluidic channel 130. This may allow for larger volumes of fluid sample to pass through the microfluidic channel 130. In an example, the depth of the microfluidic channel 130 may be increased due to the presence of these additional inertial pumps 120, but the width of the microfluidic channel 130 may be kept the same. As a particular example, the width of the microfluidic channel 130 may be maintained at 20 micrometers wide while the depth of the microfluidic channel 130 is 40, 60, or 80 micrometers, or larger.

FIG. 12 also depicts an example with a barrier 1222 between adjacent inertial pumps 120. The barrier 1222 helps to guide bubble formation when the inertial pumps 120 are activated. On the right side, the inertial pumps 120 are without a barrier 1222. Both configurations can be used for pumping liquid in a deeper microfluidic channel 130.

In the description above, for purposes of explanation, specific details are set forth in order to provide a thorough understanding of the disclosure. It will be apparent, however, to one skilled in the art that examples consistent with the present disclosure may be practiced without these specific details. Reference in the specification to “an implementation,” “an example” or similar language means that a particular feature, structure, or characteristic described in connection with the implementation or example is included in at least that one implementation, but not necessarily in other implementations. The various instances of the phrase “in one implementation” or similar phrases in various places in the specification are not necessarily all referring to the same implementation.

The preceding description has been presented only to illustrate and describe examples of the principles described. This description is not intended to be exhaustive or to limit these principles to any precise form disclosed. Many modifications and variations are possible in light of the above teaching.

1. A device for conducting a reaction with a fluid sample, the device comprising:
a microfluidic channel;
a number of heating elements along the microfluidic channel; and
an inertial pump at each of opposite ends of the microfluidic channel to oscillate the fluid sample along the microfluidic channel.

2. The device of claim 1, further comprising a fluid reservoir at each end of the microfluidic channel, wherein one of the inertial pumps is located at each interface between the microfluidic channel and one of the two reservoirs.

3. The device of claim 1, further comprising a fluid reservoir, the microfluidic channel having opposite ends that are both in fluid connection with the fluid reservoir, wherein one of the inertial pumps is located at each interface between the microfluidic channel and the reservoir.

4. The device of claim 1, wherein each heating element comprises a thin-film resistor.

5. The device of claim 1, wherein:
the reaction is a Polymerase Chain Reaction (PCR); and
the number of heating elements is three, each heating element to maintain a different temperature in the PCR.

6. The device of claim 5, wherein:
a first heating element corresponds to a denature phase of the PCR;
a second heating element corresponds to an anneal phase of the PCR;
a third heating element corresponds to an extension phase of the PCR.

7. The device of claim 1, wherein only one heating element is disposed along the microfluidic channel.

8. The device of claim 1, further comprising an injector to insert a bubble of immiscible fluid on either side of the fluid sample in the microfluidic channel.

9. The device of claim 1, wherein each inertial pump comprises a thermal inkjet pump.

10. A method of performing a Polymerase Chain Reaction (PCR), the method comprising:
introducing a fluid sample into a microfluidic channel;
oscillating the fluid sample in two directions in the microfluidic channel; and
repeatedly heating the fluid sample with a number of heating elements disposed along the microfluidic channel.

11. The method of claim 10, further comprising separating the fluid sample with a bubble of immiscible fluid on either side of the fluid sample within the microfluidic channel.

12. The method of claim 10, further comprising:
heating a first heating element to a denature temperature;
heating a second heating element to an anneal temperature, lower than the denature temperature; and
heating a third heating element to an extension temperature between the denature and anneal temperatures.

13. The method of claim 10, further comprising:
heating a first of the heating elements to a denature temperature for a denature phase of the PCR;
heating none of the heating elements during an anneal phase of the PCR; and
heating all of the heating elements to an extension temperature during an extension phase of the PCR.

14. The method of claim 13, further comprising:
after heating the fluid sample with the first heating element at the denature temperature, turning off all the heating elements and moving the fluid sample in the microfluidic channel;
after the anneal phase of the PCR, again moving the fluid in the microfluidic channel and heating all the heating elements to the extension temperature; and
after the extension phase of the PCR, returning the fluid sample to an initial position.

15. A Polymerase Chain Reaction (PCR) device for conducting a PCR on a fluid sample, the device comprising:
a microfluidic channel;
a number of heating elements along the microfluidic channel;
an inertial pump at each of opposite ends of the microfluidic channel to oscillate the fluid sample along the microfluidic channel;
a side channel fluidly connected to the microfluidic channel for introducing the fluid sample into the microfluidic channel; and
a pump at the junction of the microfluidic channel and side channel to provide a microfluidic valve between the side channel and microfluidic channel.
